(19) United States
(12) Patent Application Publication
(10) Pub. No.: US 2023/0020930 A1
(43) Pub. Date: Jan. 19, 2023

(54) ARTIFICIAL BLOOD VESSEL AND METHOD FOR MANUFACTURING AN ARTIFICIAL BLOOD VESSEL

(71) Applicant: TERUMO KABUSHIKI KAISHA, Tokyo (JP)

(72) Inventors: Keiko KONDO, Odawara-shi (JP); Emi KIKUCHI, Hadano-shi (JP); Narumi HIRATA, Hadano-shi (JP); Taketsugu NOMURA, Saitama-shi (JP)

(21) Appl. No.: 17/956,564

(22) Filed: Sep. 29, 2022

Related U.S. Application Data
(63) Continuation of application No. PCT/JP2021/009153, filed on Mar. 9, 2021.

(30) Foreign Application Priority Data
Mar. 31, 2020 (JP) 2020-062165

(51) Int. Cl. A61L 27/50; C08L 71/02; A61L 27/34

(52) U.S. Cl. CPC A61L 27/507; A61L 27/34; C08L 71/02; A61L 2420/02; C08L 2203/02

(57) ABSTRACT

Provided is an artificial blood vessel in which flexibility and a hemostatic effect required for an implantation procedure are appropriately secured. An artificial blood vessel 1 includes a base material 11 made of a fiber having a porous structure, and a coating layer 12 formed on a surface of the base material. The coating layer contains a hydrophilic polymer and a humectant, and a weight ratio of the humectant to the hydrophilic polymer is 0.1 wt% to 40 wt%.

### CROSS-REFERENCE TO RELATED APPLICATIONS

The present disclosure is a continuation of and claims benefit to PCT/JP2021/009153 filed on Mar. 9, 2021, entitled “ARTIFICIAL BLOOD VESSEL AND METHOD FOR MANUFACTURING ARTIFICIAL BLOOD VESSEL” which claims priority to Japanese Patent Application No. 2020-062165 filed on Mar. 31, 2020. The entire disclosure of the applications listed above are hereby incorporated herein by reference, in their entireties, for all that they teach and for all purposes.

### BACKGROUND

The present disclosure relates to an artificial blood vessel and a method for manufacturing an artificial blood vessel.

A technique for plugging (sealing) an artificial blood vessel base material having a porous structure has been developed. As disclosed in Japanese Pat Application No. JP-11-99163, a preclotting technique in which blood of a patient is coagulated around an artificial blood vessel is generally known, but in recent years, a technique for performing sealing by coating a porous structure of a base material with a biological material such as gelatin or collagen has been widely used.

In Japanese Pat No. 4627978, when an artificial blood vessel containing a fiber such as a polyester fiber (e.g., polyethylene terephthalate (PET)) and coated with collagen or the like is previously impregnated with an in-vivo absorbing substance such as glycerin, polyethylene glycol (PEG), and dextrin, a hemostatic effect can be improved, drying can be prevented, and flexibility can be maintained.

The biological material such as collagen is prone to peeling and foreign matter generation by a procedure using forcipes or the like or by an external stimulus during storage. In addition, the biological material is cured by drying, and handling becomes complicated. Therefore, a sealing material that is less likely to generate a foreign matter due to an external stimulus and is less likely to cause a reduction in puncture performance due to drying as compared with such a material is needed.

Therefore, for example, it is conceivable to use a hydrophilic synthetic polymer such as polyethylene glycol as the sealing material. However, when such a polymer is applied as the sealing material to a base material as it is, the polymer becomes hard by drying, the puncture performance for the artificial blood vessel is reduced, and needling for suturing an in-vivo blood vessel and the artificial blood vessel is hindered.

### BRIEF SUMMARY

An object of the present disclosure is to provide an artificial blood vessel in which flexibility and a hemostatic effect required for an implantation procedure are appropriately secured and a method for manufacturing the artificial blood vessel.

In order to solve the problems described above, an artificial blood vessel according to the present disclosure includes a base material made of a fiber having a porous structure, and a coating layer formed on a surface of the base material. The coating layer contains a hydrophilic polymer and a humectant, and a weight ratio of the humectant to the hydrophilic polymer is 0.1 weight (wt)% to 40 wt%.

In addition, in a method for manufacturing an artificial blood vessel according to the present disclosure, a coating layer is formed by applying a sealing material containing a hydrophilic polymer and a humectant and having a weight ratio of the humectant to the hydrophilic polymer of 0.1 wt% to 40 wt%to at least a portion of a surface of a base material made of a fiber having a porous structure.

According to the present disclosure, since the weight ratio of the humectant to the hydrophilic polymer is 0.1 wt% to 40 wt%, needling for suturing an in-vivo blood vessel and the artificial blood vessel is possible, and an artificial blood vessel base material having a porous structure can be plugged.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a front view of an artificial blood vessel; and

FIG. 2 is a cross-sectional view of the artificial blood vessel along a longitudinal axis direction.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

It is with respect to the above issues and other problems that the embodiments presented herein were contemplated.

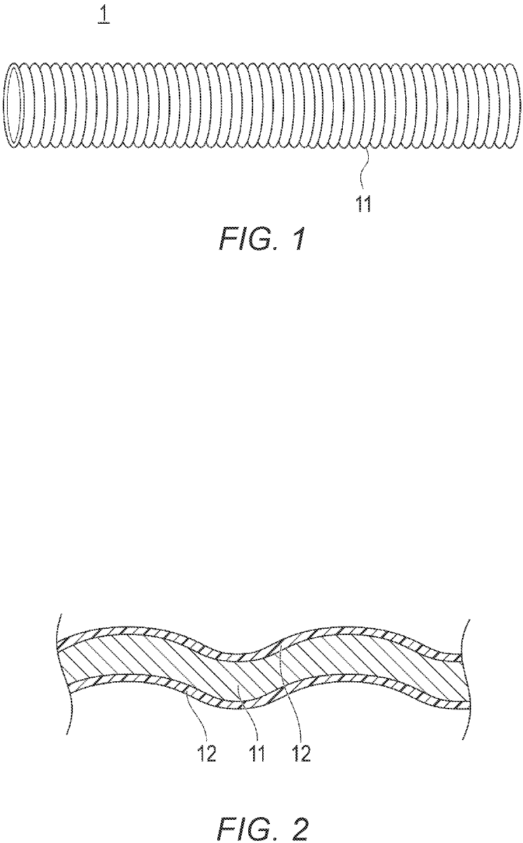

At least one embodiment of the present disclosure will be described below with reference to the accompanying drawings. FIG. 1 is a front view of an artificial blood vessel 1 according to at least one embodiment of the present disclosure.

The artificial blood vessel 1 can be used, for example, as an alternative that partially replaces an aortic arch in a surgical treatment of the aortic arch (e.g., in an aortic arch replacement surgery). However, specific applications and details of the procedure to be applied are not particularly limited.

A base material 11 of the artificial blood vessel 1 is formed of a material having flexibility and having a porous structure applicable as an alternative for a blood vessel of a living body. As such a material, for example, an artificial material such as a polyester fiber (e.g., PET), expanded polytetrafluoroethylene (ePTFE), and polyurethane can be used.

The base material 11 can be plugged by being coated, on a portion or an entirety of an inner peripheral surface, on a portion or an entirety of an outer peripheral surface, or a combination thereof, with a hydrophilic polymer described later, or a biological material such as an endothelial cell or a protein.

A size and shape of the base material 11 may be set or arranged to form a large diameter artificial blood vessel for chest suitable for replacement with the aortic arch. For example, in this case, a tube is suitable for the aortic arch by setting an outer diameter of about 12 millimeter (mm) to 30 mm, a thickness of about 0.1 mm to 1 mm, and a length of about 100 mm to 600 mm.

In addition, a bellows structure is adopted for the base material 11 so as not to collapse even when a side surface is bent during surgery. Instead of the bellows structure, a ring-shaped rib may be formed on the outer peripheral surface of the base material 11, or may be formed on a smooth surface if the base material 11 itself has a constant elastic force. In addition, the artificial blood vessel 1 may include a branch pipe branched from the base material 11 (main pipe). The number, position, inner diameter, outer diameter, and the like of the branch pipe are not particularly limited.

After the artificial blood vessel 1 is implanted, a blood vessel may be manufactured or formed in which a biological tissue enters a porous structure inside the base material 11, an inner surface and an outer surface are made of the biological tissue, and a cavity is made of an artificial material such as a polyester fiber.

FIG. 2 is a partial cross-sectional view of the artificial blood vessel 1 along a longitudinal axis direction.

The inner surface and the outer surface of the base material 11 are coated with a coating layer 12 by spraying or the like. A portion of the coating layer 12 enters between meshes of the base material. Accordingly, the porous structure of the base material 11 is plugged, and a hemostatic effect of the artificial blood vessel 1 is exhibited. The coating layer 12 contains a hydrophilic polymer and a humectant. Although not illustrated, the coating layer 12 may be provided on a portion, or an entirety, of at least one of the inner surface and the outer surface of the base material 11.

The humectant in the coating layer 12 can be made of any one or more of glycerin, diglycerin, triglycerol, ethylene glycol, and/or the like.

The hydrophilic polymer in the coating layer 12 is made of a chemically synthesized polymer formed by condensation polymerization using a polymer having a polyethylene glycol (PEG) skeleton, or a biocompatible polymer such as dextrin.

In some embodiments, the hydrophilic polymer in the coating layer 12 is made of Tetra-PEG having a three-dimensional crosslinked (e.g., mesh) structure, which is generated by an A-B type interlinking reaction between two kinds of four-branched polymers having a polyethylene glycol skeleton as represented by the following Chemical Formulas 1 and 2, that is, generated by alternative condensation polymerization using functional groups in both the polymers.

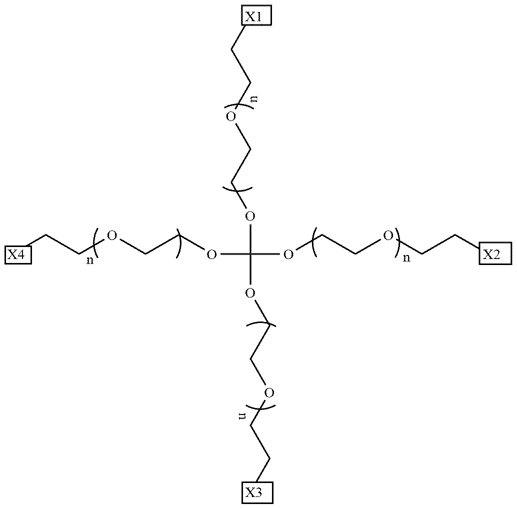

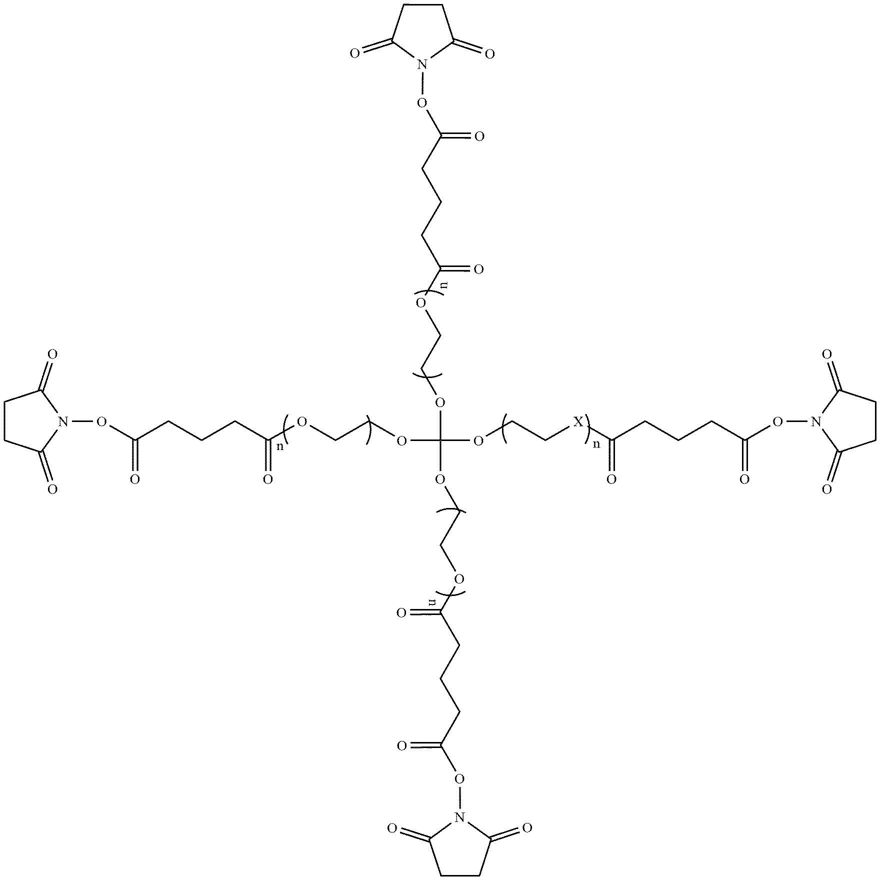

Each of functional groups X1 to X4 at a terminal in Chemical Formula 1 is any one of an amino group, a thiol group, a carboxyl group, and an aldehyde group. Among these, from a viewpoint of biological synthesis, each of the functional groups X1 to X4 is preferably either an amino group or a thiol group. The Tetra-PEG is known to have a three-dimensionally uniform mesh structure. Therefore, strength of the Tetra-PEG is higher than that of a hydrophilic polymer having an ununiform mesh structure such as gelatin, and breakage or foreign matter generation due to a procedure using forcipes or the like is less likely to occur.

However, since the Tetra-PEG has high strength and lacks flexibility, puncture resistance when passing a suture needle or the like is too large, and a suture procedure for the blood vessel and the artificial blood vessel 1 is complicated. Therefore, it is necessary to coat the coating layer 12 with a humectant made of an in-vivo absorbing substance such as glycerin to secure flexibility. On the other hand, when the coating layer 12 is coated with the humectant more than necessary, the artificial blood vessel 1 becomes too soft, and a procedure such as grasping by forcipes may be hindered, and the hemostatic effect may be reduced.

The present disclosure describes, through an example experiment, that when the coating layer 12 (e.g., sealing material) contains a hydrophilic polymer and a humectant, and a weight ratio of the humectant to the hydrophilic polymer is 0.1 wt% to 40 wt%, an artificial blood vessel 1 having high strength, and flexibility and a hemostatic effect that does not hinder the procedure can be obtained.

In addition, the present disclosure provides that when the coating layer 12 having the above weight ratio is applied to the surface of the base material 11 at an amount per unit length of the base material 11 of 0.12 grams (g)/centimeter (cm)² to 0.14 g/cm², an artificial blood vessel 1 having high strength, and flexibility and a hemostatic effect that does not hinder the procedure and reducing the foreign matter generation in the procedure can be obtained.

Hereinafter, an effect of the coating layer 12 having the above weight ratio will be described with reference to Examples and Comparative Examples.

### Needle Hole Sealing Performance Test

For artificial blood vessels in Examples and Comparative Examples, a puncture and suture operation is performed at two locations with a width of about 2 mm near a center. Thereafter, one side of the artificial blood vessel is connected to a water pressure meter, and after an air bleeding operation, the other side is sealed with forcipes. A water pressure is gradually increased, and the pressure when solution leakage occurred is recorded. A load pressure is set to a test upper limit value of 20 kilopascal (kPa) with reference to 16 kPa corresponding to 120 millimeters of mercury (mmHg) as a high value among a general normal blood pressure range.

### Piercing Resistance Test

For the artificial blood vessels in Examples and Comparative Examples, a puncture and suture operation is performed at two locations with a width of 2 mm near the center. At this time, a resistance value at the time of puncture and threading of a suture is scored by a sensitivity test. At this time, a score of the artificial blood vessel shown in Comparative Example 5 is set to 3, and 1: inferior, 2: slightly inferior, 3: equivalent, 4: slightly excellent, and 5: excellent.

### Foreign Matter Test

The artificial blood vessels in Examples and Comparative Examples are immersed in a physiological salt solution for 5 minutes and then taken out. A sealing operation is repeated five times with forcipes near the center of the artificial blood vessel taken out. The forcipes are removed, visual observation is performed, and “B” is marked when foreign matter generation due to peeling of a seal layer is confirmed, and “A” is marked when the foreign matter generation cannot be confirmed.

### Leakage Test

For the artificial blood vessels in Examples and Comparative Examples, one side is connected to a water pressure meter, and after an air bleeding operation, the other side is sealed with forcipes. A water pressure is gradually increased, and the pressure when solution leakage occurs is recorded. A load pressure is set to a test upper limit value of 16 kPa corresponding to 120 mmHg as a high value among a general normal blood pressure range.

### Manufacturing Method

As all of artificial blood vessel base materials in Examples 1 and 2 and Comparative Examples 1 to 5, a cylindrical body having an inner diameter of 8 mm and made of a polyester -based fiber woven with polyester yarns having a total fineness of 44 decitex (dtex) at a porosity of about 350 is used. The porosity is a value representing an amount of a physiological salt solution that leaks per cm² per minute when the physiological salt solution is injected into the artificial blood vessel at a pressure of 120 mmHg.

### Example 1

SUNBRIGHT PTE-100SH (manufactured by Yuka Sangyo Co., Ltd.) in which a functional group at each four-branched terminal in Tetra-PEG is a thiol group is prepared at 20% (w/v) to prepare a preparation solution A. For the preparation solution A, a mixture containing a 25 mM phosphate buffer and a 45 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 20% (w/v) to prepare a preparation solution B. For the preparation solution B, a 0.125 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight (e.g., between and/or including 1 hour and 24 hours, etc.) at room temperature (e.g., between and/or including 20° C. to 25° C., plus or minus 5° C., etc.)(state after pretreatment). After drying, the base material is immersed in a glycerin aqueous solution prepared at 25% (w/v) for 20 minutes. After 20 minutes, the artificial blood vessel base material is collected from the solution, washed with ethanol, and dried in an oven at 100° C. for 1 h (state after final treatment). This sample is referred to as Example 1.

### Example 2

SUNBRIGHT PTE-100SH (manufactured by Yuka Sangyo Co., Ltd.) in which a functional group at each four-branched terminal in Tetra-PEG is a thiol group is prepared at 20% (w/v) to prepare a preparation solution A. For the preparation solution A, a mixture containing a 25 mM phosphate buffer and a 45 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 20% (w/v) to prepare a preparation solution B. For the preparation solution B, a 0.125 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight at room temperature (e.g., between and/or including 1 hour to 24 hours at 20° C. to 25° C., plus or minus 5° C., etc.). After drying, the base material is immersed in a glycerin aqueous solution prepared at 50% (w/v) for 20 minutes. After 20 minutes, the artificial blood vessel base material is collected from the solution, washed with ethanol, and dried in an oven at 100° C. for 1 h. This sample is referred to as Example 2.

### Comparative Example 1

SUNBRIGHT PTE-100SH (manufactured by Yuka Sangyo Co., Ltd.) in which a functional group at each four-branched terminal in Tetra-PEG is a thiol group is prepared at 20% (w/v) to prepare a preparation solution A. For the preparation solution A, a mixture containing a 25 mM phosphate buffer and a 45 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 20% (w/v) to prepare a preparation solution B. For the preparation solution B, a 0.125 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight at room temperature (e.g., between and/or including 1 hour to 24 hours at 20° C. to 25° C., plus or minus 5° C., etc.). After drying, the base material is immersed in a glycerin aqueous solution prepared at 1% (w/v) for 20 minutes. After 20 minutes, the artificial blood vessel base material is collected from the solution, washed with ethanol, and dried in an oven at 100° C. for 1 h. This sample is referred to as Comparative Example 1.

### Comparative Example 2

SUNBRIGHT PTE-100SH (manufactured by Yuka Sangyo Co., Ltd.) in which a functional group at each four-branched terminal in Tetra-PEG is a thiol group is prepared at 20% (w/v) to prepare a preparation solution A. For the preparation solution A, a mixture containing a 25 mM phosphate buffer and a 45 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 20% (w/v) to prepare a preparation solution B. For the preparation solution B, a 0.125 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight at room temperature (e.g., between and/or including 1 hour to 24 hours at 20° C. to 25° C., plus or minus 5° C., etc.). After drying, the base material is immersed in a glycerin aqueous solution prepared at 75% (w/v) for 20 minutes. After 20 minutes, the artificial blood vessel base material is collected from the solution, washed with ethanol, and dried in an oven at 100° C. for 1 h. This sample is referred to as Comparative Example 2.

### Comparative Example 3

SUNBRIGHT PTE-100SH (manufactured by Yuka Sangyo Co., Ltd.) in which a functional group at each four-branched terminal in Tetra-PEG is a thiol group is prepared at 20% (w/v) to prepare a preparation solution A. For the preparation solution A, a mixture containing a 50 mM phosphate buffer and a 90 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 20% (w/v) to prepare a preparation solution B. For the preparation solution B, a 0.25 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight at room temperature (e.g., between and/or including 1 hour to 24 hours at 20° C. to 25° C., plus or minus 5° C., etc.). After drying, the base material is immersed in a glycerin aqueous solution prepared at 50% (w/v) for 20 minutes. After 20 minutes, the artificial blood vessel base material is collected from the solution, washed with ethanol, and dried in an oven at 100° C. for 1 h. This sample is referred to as Comparative Example 3.

### Comparative Example 4

SUNBRIGHT PTE-100SH (manufactured by Yuka Sangyo Co., Ltd.) in which a functional group at each four-branched terminal in Tetra-PEG is a thiol group is prepared at 5% (w/v) to prepare a preparation solution A. For the preparation solution A, a mixture containing a 25 mM phosphate buffer and a 45 mM sodium carbonate solution is used as a solvent. SUNBRIGHT PTE-100GS (manufactured by Yuka Sangyo Co., Ltd.) in which each four-branched terminal in Tetra-PEG is an N-hydroxy ester is prepared at 5% (w/v) to prepare a preparation solution B. For the preparation solution B, a 0.125 mM phosphate buffer is used as a solvent. The preparation solution A and the preparation solution B are mixed at 1:1, and then a non-coated artificial blood vessel base material is immersed in the mixture for 10 minutes. After 10 minutes, the artificial blood vessel base material is collected from the mixture, and dried overnight at room temperature (e.g., between and/or including 1 hour to 24 hours at 20° C. to 25° C., plus or minus 5° C., etc.). After drying, the base material is immersed in a glycerin aqueous solution prepared at 50% (w/v) for 20 minutes. After 20 minutes, the artificial blood vessel base material is collected from the solution, washed with ethanol, and dried in an oven at 100° C. for 1 h. This sample is referred to as Comparative Example 4.

### Comparative Example 5

An artificial blood vessel in which the base material is coated with a sealing material made of gelatin is referred to as Comparative Example 5.

Results regarding the needle hole sealing performance test, the piercing resistance test, the foreign matter test, and the leakage test for the artificial blood vessels in Examples 1 and 2 and Comparative Examples 1 to 5 are as shown in Table 1 below. In Table 1, weight X of humectant = sample weight in state after final treatment - sample weight in state after pretreatment, weight Y of hydrophilic polymer = sample weight in state after pretreatment -weight of base material, and weight ratio of humectant to hydrophilic polymer = X/Y (wt%). In addition, in Table 1, amount of sealing material is calculated according to the following equation. Here, a diameter of 0.8 cm is a diameter from front to back. Surface area = 0.8 ([cm]: diameter) × 3.14 × length [cm] × 2. Amount of sealing material = (coated weight - non-coated weight)/surface area.

Table 1

| | Amount of humectant | Amount of sealing material | Needle hole sealing performance test | Piercing resistance test | Foreign matter test | Leakage test |
|---|---|---|---|---|---|---|
| Example 1 | 5 wt% | 0.12 g/cm² | 20 kPa or more | 3 | A | 16 kPa or more |
| Example 2 | 21 wt% | 0.14 g/cm² | 20 kPa or more | 3 | A | 16 kPa or more |
| Comparative Example 1 | 0.09 wt% | 0.11 g/cm² | 20 kPa or more | 1 | A | 16 kPa or more |
| Comparative Example 2 | 43 wt% | 0.17 g/cm² | 10 kPa | 3 | A | 16 kPa or more |
| Comparative Example 3 | 35 wt% | 2.71 g/cm² | 20 kPa or more | 2 | B | 16 kPa or more |
| Comparative Example 4 | 7 wt% | 0.02 g/cm² | 3 kPa | 4 | A | 5 kPa |
| Comparative Example 5 | | | 16 kPa | 3 | A | 16 kPa or more |

As can be confirmed from Table 1, regarding the piercing resistance test, the foreign matter test, and the leakage test, Example 1 and Example 2 show the performance same as Comparative Example 5. Further, regarding the needle hole sealing performance test, Example 1 and Example 2 show performance higher than Comparative Example 5.

When Example 1 and Example 2 are compared, the same performance is shown regarding the needle hole sealing performance test, the piercing resistance test, the foreign matter test, and the leakage test. Therefore, if the weight ratio of the humectant is 5 wt% or more and 21 wt% or less and the amount of the sealing material per unit length is 0.12 g/cm² or more and 0.14 g/cm² or less, needle hole sealing performance, piercing resistance, foreign matter generation reduction, and sealing performance that are equal to or higher than those of the related art can be obtained.

In Comparative Example 1, the weight ratio of the humectant to the hydrophilic polymer is smaller than that in Example 1. In Comparative Example 1, the performance of the piercing resistance test is inferior to that in Comparative Example 5. Therefore, it is found by the present disclosure that securement of needling is insufficient with the weight ratio of the humectant in Comparative Example 1.

In Comparative Example 2, the weight ratio of the humectant to the hydrophilic polymer is larger than that in Example 2. In Comparative Example 2, the performance of the needle hole sealing performance test is inferior to that in Comparative Example 5. Therefore, it is found by the present disclosure that the weight ratio of the humectant in Comparative Example 2 is excessive with respect to the securement of the needle hole sealing performance.

In Comparative Example 3, the weight ratio of the amount of the humectant to the hydrophilic polymer and the amount of the sealing material per unit length are larger than those in Example 2. In Comparative Example 3, the performance of the foreign matter test is inferior to that in Comparative Example 5. That is, by increasing the amount of the sealing material, the same performance as in the related art can be obtained regarding the needle hole sealing performance test, the piercing resistance test, and the leakage test. However, generation of foreign matters increases as compared with the related art. Therefore, it is found by the present disclosure that the weight ratio of the humectant in Comparative Example 3 is not excessive with respect to the securement of the needle hole sealing performance and the like, but the amount of the sealing material is excessive with respect to the foreign matter generation.

In Comparative Example 4, the weight ratio of the amount of the humectant to the hydrophilic polymer is larger than that in Example 1, and is smaller than that in Example 2. In addition, the amount of the sealing material per unit length in Comparative Example 4 is smaller than that in Examples 1 and 2. In Comparative Example 4, the performance of the needle hole sealing performance test and the leakage test is inferior to that in Comparative Example 5. Therefore, it is found by the present disclosure that the amount of the sealing material in Comparative Example 3 is insufficient with respect to the needle hole sealing performance and the like.

Based on Table 1, when the coating layer 12 is obtained by coating the surface of the base material 11 with a sealing material containing a hydrophilic polymer and a humectant and having a weight ratio of the humectant to the hydrophilic polymer of 0.1 wt% to 40 wt%(in some embodiments, more specifically 5 wt% to 40 wt%), the present disclosure provides an artificial blood vessel 1 having high strength, and flexibility and a hemostatic effect that does not hinder the procedure, and reduces the foreign matter generation.

In addition, based on Table 1, when the coating layer 12 having the above weight ratio is provided on the surface of the base material 11 in an amount per unit area of the base material 11 of 0.05 g/cm² to 2 g/cm² (in some embodiments, more specifically 0.1 g/cm² to 2 g/cm²), the present disclosure provides an artificial blood vessel 1 having high strength, and flexibility and a hemostatic effect that does not hinder the procedure, and reduces the foreign matter generation.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Z₀).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. An artificial blood vessel comprising:
a base material comprising a fiber having a porous structure; and
a coating layer formed on a surface of the base material, wherein
the coating layer contains a hydrophilic polymer and a humectant, and a weight ratio of the humectant to the hydrophilic polymer is 0.1 wt% to 40 wt%.

2. The artificial blood vessel according to claim 1, wherein
the hydrophilic polymer is a four-branched polymer having a polyethylene glycol (PEG) skeleton.

3. The artificial blood vessel according to claim 2, wherein
the hydrophilic polymer comprises Tetra-PEG, the Tetra-PEG generated by an interlinking reaction between a first four-branched polymer represented by formula (1) and a second four-branched polymer represented by formula (2): 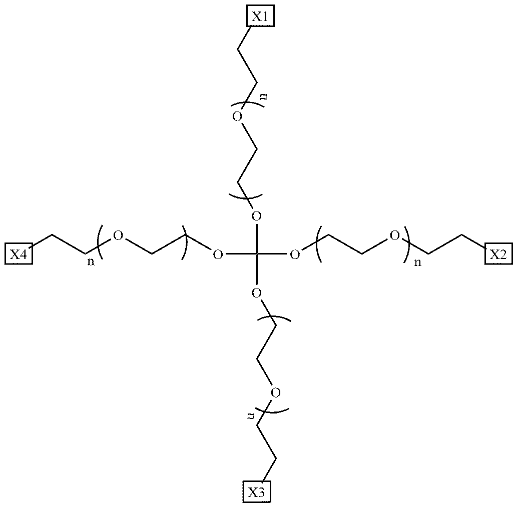 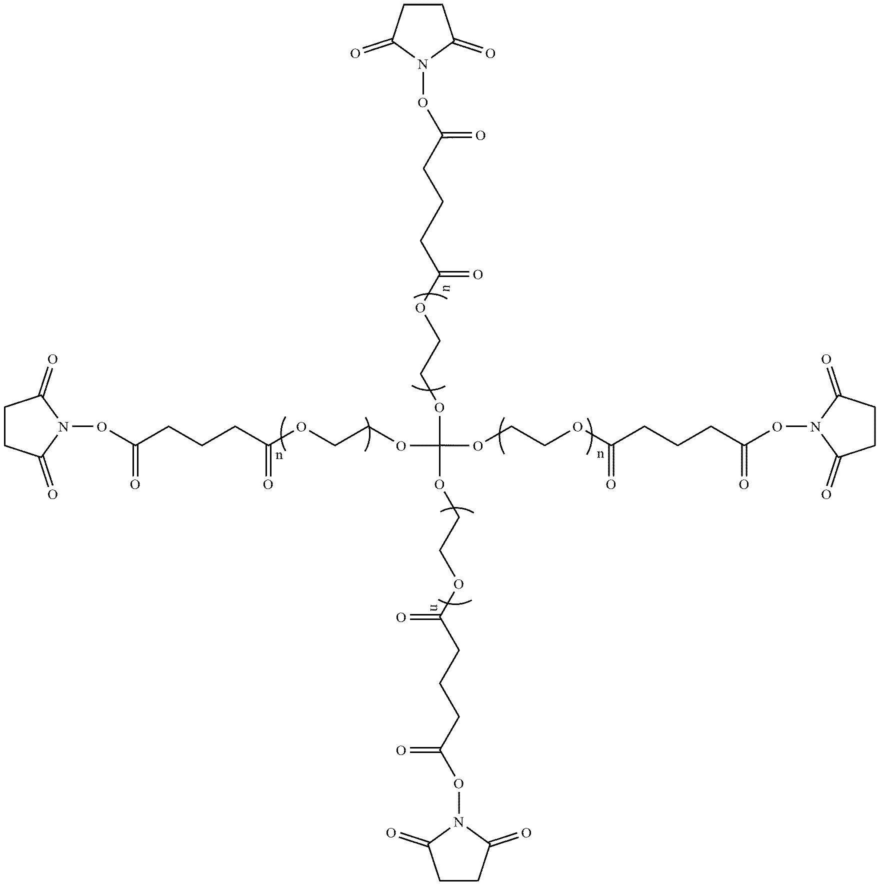
wherein each of functional groups X1 to X4 of formula (1) represents any one of an amino group, a thiol group, a carboxyl group, and an aldehyde group.

4. The artificial blood vessel according to claim 1, wherein
the humectant contains at least one of glycerin, diglycerin, triglycerol, and ethylene glycol.

5. The artificial blood vessel according to claim 1, wherein
the fiber contains at least one of polyethylene terephthalate (PET), expanded polytetrafluoroethylene (ePTFE), and polyurethane.

6. The artificial blood vessel according to claim 1, wherein
the coating layer is applied to the surface of the base material in a weight of 0.05 g/cm² to 2 g/cm² per unit area of the base material.

7. The artificial blood vessel according to claim 1, wherein
the coating layer is applied to a portion or an entirety of an outside surface of the base material.

8. The artificial blood vessel according to claim 1, wherein
the coating layer is applied to a portion or an entirety of an inside surface of the base material.

9. The artificial blood vessel according to claim 1, wherein
the base material forms a tube shape having an outer diameter between 12 mm and 30 mm, a thickness between 0.1 mm to 1 mm, and a length between 100 mm to 600 mm.

10. The artificial blood vessel according to claim 1, wherein
the base material forms a tube shape comprising a ring-shaped rib pattern, the ring-shaped rib pattern formed on at least the outside surface of the base material.

11. A method for manufacturing an artificial blood vessel, comprising:
applying, to at least a portion of a surface of a base material made of a fiber having a porous structure, a sealing material containing a hydrophilic polymer and a humectant and having a weight ratio of the humectant to the hydrophilic polymer of 0.1 wt% to 40 wt% to form a coating layer.

12. The method for manufacturing the artificial blood vessel according to claim 11, wherein applying the sealing material comprises:
applying the sealing material to at least a portion of an outside surface of the base material, at least a portion of an inside surface of the base material, or both.

13. The method for manufacturing the artificial blood vessel according to claim 11, wherein applying the sealing material comprises:
spraying the sealing material on at least the portion of the surface of the base material until the porous structure of the base material is filled with the sealing material.

14. The method for manufacturing the artificial blood vessel according to claim 11, wherein applying the sealing material comprises:
immersing the base material into the hydrophilic polymer for a first duration;
drying the base material coated with the hydrophilic polymer at a first temperature for a second duration;
immersing the base material coated with the hydrophilic polymer into the humectant for a third duration;
washing, with an alcohol, the base material coated with the hydrophilic polymer and the humectant; and
drying the washed base material coated with the hydrophilic polymer and the humectant at a second temperature for a fourth duration.

15. The method for manufacturing the artificial blood vessel according to claim 11, further comprising:
mixing a first solution and a second solution in a one-to-one ratio to form the hydrophilic polymer, wherein the first solution contains Tetra-PEG comprising a thiol group at each of the terminals of the Tetra-PEG, and wherein the second solution contains Tetra-PEG comprising an N-hydroxy ester at each of the terminals of the Tetra-PEG.

16. The method for manufacturing the artificial blood vessel according to claim 11, further comprising:
preparing a glycerin aqueous solution at 25% weight per volume, wherein the glycerin aqueous solution is the humectant.

17. The method for manufacturing the artificial blood vessel according to claim 11, further comprising:
preparing a glycerin aqueous solution at 50% weight per volume, wherein the glycerin aqueous solution is the humectant.

18. A coating layer, comprising:
a hydrophilic polymer comprising a four-branched polymer having a polyethylene glycol (PEG) skeleton; and
a humectant, wherein a weight ratio of the humectant to the hydrophilic polymer is 0.1 wt%to 40 wt%.

19. The coating layer according to claim 18, wherein
the humectant contains at least one of glycerin, diglycerin, triglycerol, and ethylene glycol.

20. The coating layer according to claim 18, wherein
the hydrophilic polymer comprises Tetra-PEG, the Tetra-PEG generated by an interlinking reaction between a first four-branched polymer represented by formula (1) and a second four-branched polymer represented by formula (2): 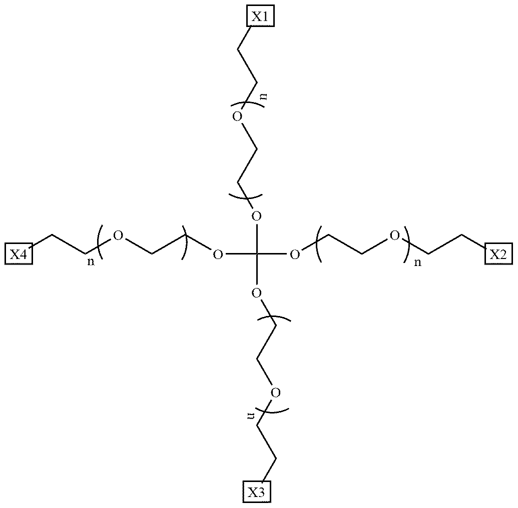 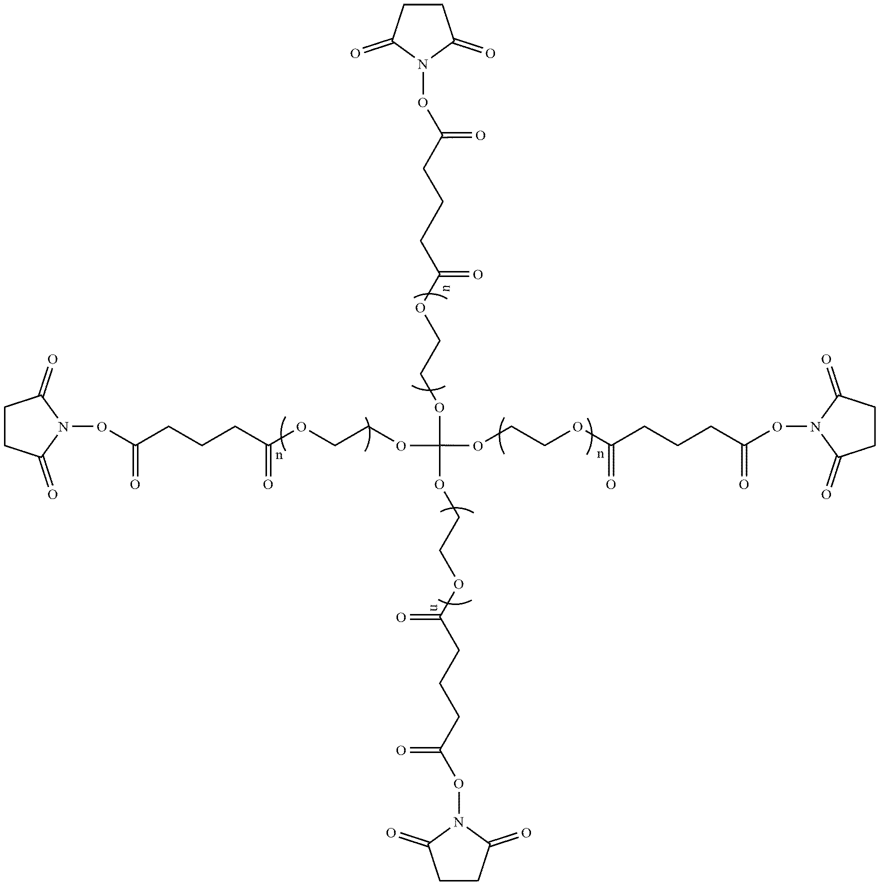
wherein each of functional groups X1 to X4 of formula (1) represents any one of an amino group, a thiol group, a carboxyl group, and an aldehyde group.
